(19) United States
(12) Patent Application Publication
(10) Pub. No.: US 2002/0034558 A1
(43) Pub. Date: Mar. 21, 2002

(54) Compositions in the form of water-in-oil emulsions and their use as cosmetics

(75) Inventors: Odile Sonneville-Aubrun, Antony (FR); Jean-Thierry Simonnet, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L' OREAL, PARIS (FR)

(21) Appl. No.: 09/848,412

(22) Filed: May 4, 2001

(30) Foreign Application Priority Data
May 5, 2000 (FR) 0005796

(51) Int. Cl. A61K 35/78; A61K 31/7024

(52) U.S. Cl. 424/776; 514/23

(57) ABSTRACT

The invention relates to a composition in the form of a water-in-oil emulsion containing at least 80% of aqueous phase and at least one glycoglyceride. The glycoglyceride may be obtained by synthesis or by extraction, and it may be, in particular, a galactolipid such as, for example, digalactosyl diglyceride or monogalactosyl diglyceride. The composition according to the invention provides a great sensation of freshness when applied to the skin, and may be used in particular in cosmetics and/or dermatology.

### CROSS REFERENCES TO RELATED APPLICATIONS

This application claims priority to French Patent Application No. 0005796, filed on May 5, 2000, and which is incorporated herein by reference in its entirety.

### BACKGROUND OF THE INVENTION

1. Field of the Invention

The present invention relates to compositions in the form of a water-in-oil (W/O) emulsion and which have a high water content and comprise at least one glycoglyceride. The present invention further relates to the uses of such compositions in particular in cosmetics and/or dermatology.

2. Discussion of the Background

It is common practice in cosmetics or dermatology to use compositions consisting of a water-in-oil (W/O) emulsion which comprises an aqueous phase dispersed in an oily phase. These emulsions comprise an oily continuous phase and thus make it possible to form on the surface of the skin a lipid film which prevents transepidermal water loss and protects the skin against external attack. These emulsions are particularly suitable for protecting and nourishing the skin, and in particular for treating dry skin. In addition, the lipid film formed at the surface of the skin may also increase the permanence of sunscreens. These emulsions also have the advantage of allowing the protection and transport of oxidation-sensitive hydrophilic active agents.

Inverse emulsions, i.e. water-in-oil emulsions, are usually stabilized with lipophilic surfactants, which are usually chosen from alkyl derivatives of polyglycerol, polyoxyethylenated alkyl derivatives, alkyl derivatives of sorbitan and metal salts of fatty aids. Generally, the aqueous-phase content of these emulsions must remain less than 80% of the weight of the composition, and the surfactant content must be relatively high, in order for the emulsions obtained to be of acceptable stability, i.e. without phase separation or release of oil and/or of water over time.

Typically, on account of the high surfactant content and the limited water content, the formulations obtained are greasy, heavy and sticky. Furthermore, the surfactant content may give rise to tolerance problems, especially for individuals with sensitive skin.

Moreover, to improve the stability of these emulsions, it is generally necessary to increase the viscosity of the oily phase by adding a structuring agent, and, for example, either waxes as disclosed, for example, in published German patent application DE-A-3 430 256, or oily gelling agents as disclosed, for example, in published European patent application EP-A-795 321, or modified clays as disclosed, for example, in published European patent application EP-A-331 833. However, on account of the addition of a structuring agent, the thus-obtained compositions lack freshness and lightness, and they give a greasy sensation when applied to the skin.

To overcome these drawbacks of inverse emulsions with a high aqueous-phase content and a low surfactant content, use has been made of silicone surfactants such as alkylmethicone copolyols and alkyldimethicone copolyols, as disclosed in published German patent application DE-A-19 826 750, or alternatively such as dimethicone copolyols, as disclosed in published European patent application EP-A- 970 682.

However, although these silicone surfactants can improve the cosmetic properties of water-in-oil emulsions, and also their stability, the feel of the compositions containing them remains characteristic of silicones, i.e. slippery and with little emollient nature. Moreover, in a water-in-oil emulsion containing a silicone surfactant, it is generally necessary for the silicone oils to constitute the majority of the oily phase, which limits the choice of constituents of the oily phase.

Furthermore, published German patent application DE-A-19 850 758 discloses inverse emulsions with a high water content based on a dipolyhydroxyacyl/polyalkylene glycol triblock polymer surfactant such as Arlacel P135. Although this type of surfactant makes it possible to obtain fluid emulsions with a high water content, it does not ensure a sufficient stability, over time and at temperature.

It is also possible to prepare inverse emulsions with a high water content containing natural phospholipids, as disclosed in published French patent applications FR-A-2 777 180, FR-A-2 777 181, FR-A-2 777 194, and FR-A-2 777 195. However, the oily phase of the compositions disclosed in these documents is limited to restricted mixtures of oils. Furthermore, it is known by those skilled in the art that phospholipids have limited stability to oxidation and that this causes a destabilization of the support containing them.

There is thus a need for water-in-oil emulsions which do not have the drawbacks of the prior art, i.e. which have a light, fresh feel without a “silicone” sensation, and which moreover have a high aqueous-phase content and which are stable, even in the absence of oily-phase structuring agents. There also remains a need for cosmetic and dermatological compositions which contain such a water-in-oil emulsions and for methods of treating the skin with such a composition.

### SUMMARY OF THE INVENTION

Accordingly, it is one object of the present invention to provide novel water- in-oil emulsions.

It is another object of the present invention to provide novel water-in-oil emulsions which contain a high water content.

It is another object of the present invention to provide novel water-in-oil emulsions which exhibit a high stability.

It is another object of the present invention to provide novel water-in-oil emulsions which contain a high water content.

It is another object of the present invention to provide novel water-in-oil emulsions which do not have a “silicone” feel.

It is another object of the present invention to provide novel compositions which contain such a water-in-oil emulsion.

It is another object of the present invention to provide novel methods of treating the skin by applying such a composition to the skin.

These and other objects, which will become apparent during the following detailed description, have been achieved by the inventors' surprising discovery that the use of glycoglycerides makes it possible to prepare water-in-oil emulsions, which are rich in aqueous phase and stable on storage, with a consistency ranging from that of a fluid milk to a compact cream.

Thus, in a first embodiment, the present invention provides novel water-in-oil emulsions, which comprise an aqueous phase dispersed in an oily phase, wherein the emulsion comprises at least one glycoglyceride and wherein the aqueous phase represents at least 80% by weight relative to the total weight of the composition.

In a second embodiment, the present invention provides novel compositions, which comprise, in a physiologically acceptable medium, an aqueous phase dispersed in an oily phase, wherein the composition comprises at least one glycoglyceride and wherein the aqueous phase represents at least 80% by weight relative to the total weight of the composition.

In a third embodiment, the present invention provides dermatological and/or cosmetic compositions, which comprise, in a physiologically acceptable medium, an aqueous phase dispersed in an oily phase, wherein the composition comprises at least one glycoglyceride and wherein the aqueous phase represents at least 80% by weight relative to the total weight of the composition.

In a fourth embodiment, the present invention provides a method for treating the skin by applying to the skin a dermatological and/or cosmetic composition, which comprises, in a physiologically acceptable medium, an aqueous phase dispersed in an oily phase, wherein the composition comprises at least one glycoglyceride and wherein the aqueous phase represents at least 80% by weight relative to the total weight of the composition.

### DETAILED DESCRIPTION OF THE PREFERRED EMBODIMENTS

Thus, in a first embodiment, the present invention provides novel water-in-oil emulsions, which comprise an aqueous phase dispersed in an oily phase, wherein the emulsion comprises at least one glycoglyceride and wherein the aqueous phase represents at least 80% by weight relative to the total weight of the composition, while in a second embodiment, the present invention provides compositions comprising, in a physiologically acceptable medium, an aqueous phase dispersed in an oily phase, characterized in that it comprises at least one glycoglyceride and in that the aqueous phase represents at least 80% by weight relative to the total weight of the composition.

The glycoglycerides contained in the composition of the present invention are esters containing one or two alkyl and/or alkenyl chains and may be obtained by extraction or by synthesis. Those obtained by extraction are extracted in particular from plants. The synthetic products may be obtained in particular by esterifying a sugar comprising at least one carboxylic acid function with a monoglyceride diglyceride. The sugars concerned are oses (monosaccharides), oligoholosides (oligosaccharides) and polyholosides (polysaccharides) that are highly depolymerized, with a molecular mass preferably of less than 2,000 g/mol. Non- limiting examples of these sugars which may be mentioned include glucose, sucrose, trehalose, sorbitol, lactose, fructose, xylose, and maltose.

The alkyl and/or alkenyl chains of the glycoglyceride preferably comprise from 10 to 22 carbon atoms, such as the alkyl and alkenyl chains contained in the carboxylic acids chosen, for example, from capric acid, lauric acid, myristic acid, palmitic acid, stearic acid, arachidic acid, behenic acid, palmitoleic aicd, oleic acid, linoleic acid, linolenic acid, gadoleic acid, arachidonic acid, and erucic acid.

According to one preferred embodiment of the invention, the glycoglycerides used are galactolipids.

Glycoglycerides are known compounds. Thus, published European patent applications EP-A-9842 and EP-A-249 561 disclose the use of galactolipids to prepare liposomes, and WO-A-95/20945 discloses the use of galactosyl diglycerides in combination with non-polar lipids such as triacylglycerol, to prepare lipophilic supports which may be used in the pharmacy, cosmetic and agrifood sectors. The supports disclosed in published PCT patent application WO-A-95/20945 may be suspensions obtained by adding increasing amounts of water to an oily phase containing a digaalactosyl-diglyceride. However, the water-in-oil emulsions disclosed in published PCT patent application WO-A-95/20945 contain relatively low amounts of aqueous phase, much less than 80% of the weight of the composition, with large amounts of galactolipids (20%). In contrast, the Applicant has found, entirely unexpectedly, that it is possible to use galactolipids to obtain water-in-oil emulsions that are rich in aqueous phase and rich in water, in order to obtain fresh, light textures, and to do so with amounts of galactolipids that are much less than those disclosed in published PCT patent application WO-A-95/20945. Despite the large aqueous-phase content (and the large amount of water) and the small amount of galactolipids, the W/O emulsions obtained are stable, which is entirely surprising. There was nothing to suggest that such emulsions could be obtained by using galactolipids.

In the compositions of the present invention, the expression “physiologically acceptable medium” means a non-toxic medium which may be applied to the skin (including the inside of the eyelids), the hair, the nails or the lips of human beings.

Despite the large amount of aqueous phase, the compositions of the invention are stable over time. The term “stable over time” means that the emulsion does not undergo phase separation and does not release oil or water after 2 months of storage at a temperature of from 4° C. to 45° C. In addition, the compositions of the present invention provide a great sensation of freshness on the skin.

The compositions according to the present invention comprise at lest 80% by weight of aqueous phase relative to the total weight of the composition, preferably at least 82% by weight, and better still 85% by weight of the total weight of the composition. The aqueous phase may constitute up to 98% of the total weight of the composition. The expression “aqueous phase” means herein everything which constitutes the dispersed phase, i.e. the water, the solvents and the water-soluble or water-dispersible additives. Solvents which may be mentioned include, for example, linear or branched monoalcohols containing from 1 to 8 carbon atoms, for instance ethanol, propanol, isopropanol, butanol or isobutanol; polyethylene glycols containing from 4 to 80 ethylene oxides; and polyols, for instance glycerol, propylene glycol, isoprene glycol and butylene glycol. Water-soluble additives which may be mentioned, for example, include mono-, di- or trivalent mineral salts; water-soluble W screening agents such as Mexoryl SX and Eusolex 232; texture agents such as, for example, xanthan gums, cellulose derivatives, guars, carbomers, acrylamide derivatives such as polyacrylamides, and acrylic derivatives; and sugars such as glucose, sucrose, trehalose, sorbitol, lactose, fructose, xylose or maltose. Water- dispersible additives which may be mentioned, for example, include mineral or organic particles such as water-dispersible nanotitanias, such as Mirasun TiW60 from the company Rhodia, Tioveil AW-N from the company Uniqema, nanozinc oxides, clays such as Laponite XLS from the company Laporte, and latices such as Avalure AC- 115 from the company Goodrich.

According to one preferred embodiment of the invention, water constitutes at least 70 % by weight, preferably at least 75 % by weight, and better still 80 % by weight of the total weight of the composition.

As glycoglycerides which may be used in the composition of the invention, mention may be made in particular of galactolipids and in particular of digalactosyl diglyceride or 1-O-[6-O-(α-D-galactopyranosyl)-β-D-galactopyranosyl]-2,3-di-O-acyl-D-glyceritol (DGDG) which may be in hydrogenated or nonhydrogenated form; monogalactosyl diglyceride (MGDG) and mixtures thereof. These compounds exist in the natural state in plants and more particularly in plant membranes. They may be obtained by extraction from these plants, and, for example from cereals or spinach. They are disclosed, for example, in published European patent applications EP-A-9842 and EP-A-249 561, which are incorporated herein by reference. The DGDG and MGDG supplied by the companies Serdary Research Laboratories or Doosan may be used in particular. DGDG may also be prepared from fresh spinach leaves, according to the process of Example 1 of published European patent application EP-A-249 561, or from cereals according to the process of published European patent application EP-A-9842.

DGDG has the structure (I) below:

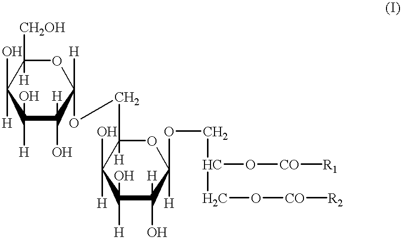

in which R₁ and R₂ may be identical or different and represent an alkyl and/or alkenyl residue containing from 10 to 22 carbon atoms, such as the alkyl and alkenyl groups in capric acid, lauric acid, myristic acid, palmitic acid, stearic acid, arachidic acid, behenic acid, palmitoleic acid, oleic acid, linoleic acid, linolenic acid, gadoleic acid, arachidonic acid, or erucic acid.

These galactolipids may be used in pure for or as mixtures containing them in larger or smaller proportions. Mixtures containing at least 10% of DGDG relative to the polar lipids of the said mixture are preferably used.

The amount of glycoglyceride(s) in the composition of the invention preferably ranges from 0.1% to 10% by weight, better still from 0.1% to 5% by weight, and more preferably from 0.5% to 5% by weight relative to the total weight of the composition.

The glycoglycerides are preferably introduced into the oily phase of the emulsion.

The glycoglycerides may be used alone or as a mixture with one or more surfactants commonly used to prepare water-in-oil emulsions. As surfactants which may be used, mention may be made, for example, of alkyl esters or ethers of sorbitan, of glycerol or of sugars, and alkyldimethicone copolyols.

When the composition of the invention contains surfactants other than glycoglycerides, these surfactants are present in an amount ranging from 0. 1% to 50% by weight relative to the weight of glycoglycerides or of the mixture of lipids containing glycoglycerides.

The oily phase of the composition according to the invention may comprise oils and fatty substances of any kind that are known to those skilled in the art, for example oils of plant origin jojoba oil, avocado oil, sesame oil, sunflower oil, corn oil, soybean oil, safflower or grape pip oil), mineral oils (liquid petroleum jelly, hydrogenated or non-hydrogenated isoparaffins or isohexadecane), synthetic oils (parleam, isopropyl myristate, cetearyl octanoate, polyisobutylene, ethylhexyl palmitate or alkyl benzoate), volatile or non-volatile silicone oils such as polydimethylsiloxanes (PDMSs) and cyclodimethylsiloxanes or cyclomethicones, and fluoro oils or fluorosilicone oils, as well as mixtures of these oils.

The oily phase may also contain other fatty constituents such as fatty alcohols, for instance stearyl alcohol, cetyl alcohol and cetearyl alcohol, or fatty acids; waxes; silicone gums; oily gelling agents such as, for example, the alkyl guars from the company Hercules; mineral or organic particles such as, for example, hydrophobic modified titanium or nanotitanium oxides such as MT-100-Z from the company Tayca; hydrophobic modified pigments; methyl methacrylate copolymer particles such as Polytrap 6035 from the company APS. The oily phase of the composition may also contain liposoluble organic sunscreens such as, for example, Parsol 1789, Parsol MCX, Uvinul N539 or Mexoryl XL.

According to one particular embodiment of the invention, the oily phase comprises at least one oil chosen from mineral oils, synthetic oils and volatile silicone oils, and mixtures thereof, and in particular at least one oil chosen from parleam oil, isohexadecane and cyclomethicones, and mixtures thereof.

The amount of oily phase in the composition of the invention, including the amount of galactolipids, may range from 0.5% to 20% by weight and preferably from 2% to 18% by weight relative to the total weight of the composition.

The compositions according to the invention may be more or less fluid, and they may thus be in the form of a milk or in the form of a more or less thick cream. Thus, another advantage of the present invention is the ability to obtain W/O emulsions of low viscosity which are stable, these being emulsions that are particularly difficult to obtain. The viscosity of the emulsions of the present invention may thus vary within a wide range and may range in particular from 1 poise (0.1 Pa·s) to 150 poises (15 Pa·s), these viscosities being measured at about 25° C. using a “Rheomat 180” viscometer which is generally fitted with a No. 2 rotor for viscosities in the range from 0.02 Pa·s to 0.7 Pa·s, a No. 3 rotor for viscosities in the range from 0.2 Pa·s to 4 Pa·s, and a No. 4 rotor for viscosities in the range from 2 Pa·s to 23 Pa·s.

The compositions of the invention are prepared according to the usual methods for preparing a water-in-oil emulsion: after the aqueous and oily phases have been prepared, the aqueous phase is gradually incorporated into the oily phase comprising the glycoglycerides, with stirring. The emulsion is generally prepared at a temperature ranging from about 20° C. to 60° C.

The compositions according to the present invention contain a physiologically acceptable medium and may be applied in a large number of treatments, especially cosmetic treatments of the skin, including the scalp, the hair, the nails and/or mucous membranes, in particular to care for, cleanse and/or make up and/or provide antisun protection to the skin, the hair and/or the lips or mucous membranes, as well as to prepare a cream for treating the skin, more particularly dry skin, on account of the presence of an outer oily phase.

In another embodiment, the present invention provides the cosmetic use of the compositions as defined above, to treat, protect, care for, remove make-up from and/or cleanse the skin, the lips and/or the hair, and/or to make up the skin and/or the lips.

In another embodiment, the present invention provides a cosmetic method for treating the skin, including the scalp, the hair and/or the lips, characterized in that a composition as defined above is applied to the skin, the hair and/or the lips.

In another embodiment, the present invention provides the use of the composition as defined above to manufacture a cream for treating dry skin.

The composition of the invention may also contain adjuvants that are common in cosmetics and/or dermatology, such as electrolytes, for instance magnesium sulphate; active agents; preserving agents; solvents; fragrances; fillers; bactericides; odor absorbers; dyestuffs; and lipid vesicles. The amounts of these various adjuvants are those conventionally used in the field under consideration, and are, for example, from 0.01% to 20% by weight of the total weight of the composition. Depending on their nature, these adjuvants may be introduced into the fatty phase or into the aqueous phase.

Active agents which may be mentioned in particular, in addition to those indicated above, include moisturizers and, for example, protein hydrolysates and polyols such as glycerol, glycols, for instance polyethylene glycols, and sugar derivatives; natural extracts; procyannidol oligomers; vitamins; urea; caffeine; depigmenting agents such as kojic acid and caffeic acid; β-hydroxy acids such as salicylic acid and its derivatives; α-hydroxy acids such as lactic acid and glycolic acid; retinoids such as carotenoids; screening agents; hydrocortisone; DHEA; melatonin; extracts of algae, of fungi, of plants, of yeasts or of bacteria; hydrolyzed, partially hydrolyzed or nonhydrolyzed proteins, and enzymes, and mixtures thereof.

The active agents may be present, for example, in a concentration ranging from 0.01% to 20% by weight, preferably from 0.1% to 5% by weight and better still from 0.5% to 3% by weight of the total weight of the composition.

As noted above, the present compositions may be used for treating, protecting, caring for, removing make-up from and/or cleansing the skin, the lips and/or the hair, and/or of making up the skin and/or the lips by applying the composition to the skin, lips, and/or hair. When using the present composition in such a manner, the composition is suitably applied to the skin, lips, and/or hair in an amount of 0.1 to 5 mg/cm², preferably 0.5 to 4 mg/cm², more preferably 1 to 3 mg/cm² (these numbers being approximate). The amount is generally 2 mg/cm^{2.}

Other features of the invention will become apparent in the course of the following descriptions of exemplary embodiments which are given for illustration of the invention and are not intended to be limiting thereof.

### EXAMPLES

In the following examples, and throughout this specification, all parts and percentages are by weight, and all temperatures are in degrees Celsius, unless expressly stated to be otherwise.

Example 1: Moisturizing milk

| A. Oily phase: | |
|---|---|
| Galactolipids (DGDG supplied by the company | 2.5% |
| Doosan) | |
| Isohexadecane | 7.7% |
| Parleam oil | 5.6% |
| Cyclomethicone | 3.9% |
| Preserving agents | 0.1% |
| B. Aqueous phase: | |
| MgSO₄.7H₂O | 0.8% |
| Water | 78.6% |
| Preservatives | 0.6% |

A white milk with an equivalent viscosity of 14.9 poises (1.49 Pa·s) (rotor No. 3, 200s¹), which is stable for at least two months from 4 to 45° C., is obtained.

Example 2: Dry-skin cream

| A. Oily phase: | |
|---|---|
| Galactolipids (DGDG supplied by the company | 2.5% |
| Doosan) | |
| Isohexadecane | 2.9% |
| Parleam oil | 2.2% |
| Cyclomethicone | 1.48% |
| Preserving agents | 0.1% |
| BHT (antioxidant) | 0.07% |
| B. Aqueous phase: | |
| MgSO₄.7H₂O | 0.9% |
| Water | 88.5% |
| Preservatives | 0.6% |

A soft cream with an equivalent viscosity of 49.7 poises (4.97 Pa·s) (rotor No. 3, 200 s¹), which is stable for at last two months from 4 to 45° C., is obtained.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A water-in-oil emulsion, comprising an aqueous phase dispersed in an oily phase, and at least one glycoglyceride, wherein said aqueous phase is at least 80% by weight of the total weight of the composition.

2. A composition comprising, in a physiologically acceptable medium, an aqueous phase dispersed in an oily phase, wherein said composition comprises at least one glycoglyceride and said aqueous phase represents at least 80% by weight of the total weight of said composition.

3. The composition according to claim 2, which comprises at least 70% of water relative to the total weight of the composition.

4. The composition according to claim 2, wherein said glycoglyceride is obtained by synthesis or by extraction.

5. The composition according to claim 2, wherein said glycoglyceride is extracted from a plant.

6. The composition according to claim 2, wherein said glycoglyceride is obtained by esterifying a sugar comprising at least one carboxylic acid function with a monoglyceride or a diglyceride.

7. The composition according to claim 2, wherein said glycoglyceride is a galactolipid.

8. The composition according to claim 2, wherein said galactolipid is selected from the group consisting of hydrogenated and non-hydrogenated digalactosyl diglyceride and monogalactosyl diglyceride, and mixtures thereof.

9. The composition according to claim 2, wherein said glycoglyceride is present in an amount of from 0.1% to 10% by weight relative to the total weight of the composition.

10. The composition according to claim 2, wherein said aqueous phase is present in an amount ranging from 80% to 98% by weight relative to the total weight of the composition.

11. The composition according to claim 2, wherein said oily phase is present in an amount ranging from 0.5% to 20% by weight relative to the total weight of the composition.

12. The composition according to claim 2, wherein said oily phase comprises at least one oil selected from the group consisting of mineral oils, synthetic oils, volatile silicone oils, and mixtures thereof.

13. The composition according to claim 2, wherein said oily phase comprises at least one oil selected from the group consisting of parleam oil, isohexadecane, and cyclomethicones, and mixtures thereof.

14. A method of treating, protecting, caring for, removing make-up from and/or cleansing the skin, the lips and/or the hair, and/or of making up the skin and/or the lips, comprising applying to the skin, lips, and/or hair a composition comprising, in a physiologically acceptable medium, an aqueous phase dispersed in an oily phase,
wherein said composition comprises at least one glycoglyceride and said aqueous phase represents at least 80% by weight of the total weight of said composition.

15. The method according to claim 14, wherein said glycoglyceride is a galactolipid.

16. The method according to claim 14, wherein said galactolipid is selected from the group consisting of hydrogenated and non-hydrogenated digalactosyl diglyceride and monogalactosyl diglyceride, and mixtures thereof.

17. The method according to claim 14, wherein said glycoglyceride is present in an amount of from 0.1% to 10% by weight relative to the total weight of the composition.

18. A method of treating dry skin, comprising applying to the skin a composition comprising, in a physiologically acceptable medium, an aqueous phase dispersed in an oily phase, wherein said composition comprises at least one glycoglyceride and said aqueous phase represents at least 80% by weight of the total weight of said composition.

19. The method according to claim 18, wherein said glycoglyceride is a galactolipid.

20. The method according to claim 18, wherein said galactolipid is selected from the group consisting of hydrogenated and non-hydrogenated digalactosyl diglyceride and monogalactosyl diglyceride, and mixtures thereof.

21. The method according to claim 18, wherein said glycoglyceride is present in an amount of from 0.1% to 10% by weight relative to the total weight of the composition.
